(19) United States
(12) Patent Application Publication
(10) Pub. No.: US 2005/0038343 A1
(43) Pub. Date: Feb. 17, 2005

(54) Apparatus and method for locating a bifurcation in an artery

(75) Inventors: Pei-Jie Cao, Fremont, CA (US); Ricardo G. Hahn, Sierra Madre, CA (US); K. Kirk Shung, Monterey Park, CA (US); Jacques D. Barth, Santa Monica, CA (US); Lynda Knox, Long Beach, CA (US)

Correspondence Address: MCDERMOTT, WILL & EMERY;Suite 3400, 2049 Century Park East, Los Angeles, CA, 90067, US

(73) Assignee: Alfred E. Mann Institute for Biomedical Research at the University of Southern California

(21) Appl. No.: 10/888,037

(22) Filed: Jul. 9, 2004

Related U.S. Application Data
(60) Provisional application No. 60/486,545, filed on Jul. 10, 2003.

(51) Int. Cl. A61B 8/06

(52) U.S. Cl. 600/454

(57) ABSTRACT

A method and apparatus are described for locating a bifurcation of an artery. An elongated probe having a plurality of ultrasonic transducers, and an indicator attached to each transducer, is moved along the artery. Each transducer transmits ultrasound signals to the portion of the anatomy located underneath the transducer, and receives echo signals reflected from the portion of the anatomy. The echo signals are analyzed to detect any presence of blood flow. Each indicator generates output reference signals, for example light or sound, only if blood flow is detected by its associated transducer. The bifurcation is located by observing when a two-group pattern of output reference signals from the indicators merges into a single-group pattern of output reference signals. A single signaling device may be used instead of a plurality of indicators. Once the bifurcation is located, the probe may be switched into a measuring mode, in which the thickness of intima media is measured at the bifurcation.

### CROSS REFERENCE TO RELATED APPLICATIONS

This application claims the benefit of priority under 35 U.S.C. §119(e) from co-pending, commonly owned U.S. provisional patent application, Ser. No. 60/486,545, filed on Jul. 10, 2003, entitled “An Ultrasound Method For Carotid Intima-Media Thickness Measurement.” The entire content of this provisional application is incorporated herein by reference.

### BACKGROUND

The measurement of intima-media thickness (IMT) in a carotid artery can be useful in medical applications, for example by defining the extent and severity of carotid disease. It has been demonstrated that IMT measurements tend to correlate well with the extent and severity of carotid disease.

By acquiring and processing images of the wall of the common carotid artery, and measuring the thickness of the rear wall, useful information regarding possible indications of atherosclerotic cardiovascular disease can be obtained. Such a procedure has become known as an IMTHeartScan or an IMTHeakthScan. The images may be digitized before being interpreted. The measurements may indicate the minimal, maximal and the average thickness of the rear wall of the common carotid artery.

A known technique for measuring the thickness of the intima-media in the carotid artery involves the analysis of echo signals, i.e. the analysis of the ultrasonic signals that have been reflected from the tissue under examination. To accomplish this, one or more ultrasonic transducers may be placed on the surface of the skin of the neck about 0.25 cm below the location at which the carotid artery bifurcates into two smaller arteries, i.e. below the bifurcation or “Y-junction” of the carotid artery in the neck.

In order to carry out IMT measurements, therefore, a technician may need to position a probe just below a bifurcation in the carotid artery, so that the thickness of intima-media can be measured at this location in accordance with known techniques. Unfortunately, technicians may often have difficulty positioning the ultrasonic transducer at this exact location because the bifurcation in the carotid artery is usually not visible from the outside of the body.

One approach to overcoming this problem has been to use the same ultrasonic probe, used to make IMT measurements, to locate the bifurcation or “Y” junction. In this approach, the ultrasonic probe is switched into a different mode, in which the probe is operable to generate signaling information from which a two dimensional image of the tissue beneath the probe can be created. Unfortunately, it is often difficult for technicians to decipher the “Y” junction or bifurcation from this two-dimensional tissue image. Also, complex and costly image processing tools are necessary in order to generate these ultrasound images.

### SUMMARY

An apparatus and method are described for locating a bifurcation of an artery that has a single branch on one side of the bifurcation, and separates into at least a first branch and a second branch on another side the bifurcation. The artery may be the carotid artery, for example. The apparatus is inexpensive and easy to operate.

The apparatus includes an elongated probe having a plurality of ultrasound transducers, which generate ultrasound signals when provided with electric energy, and which transmit the ultrasound signals onto a medium, e.g. a tissue under examination. The transducers also receive reflected signals that have been reflected back from the medium, and convert the reflected signals into electric signals that can be analyzed by a signal analyzer.

The signal analyzer analyzes the reflected signals received by each transducer, so as to determine whether blood flow within the medium can be detected from the received signals. In one embodiment, blood flow may be detected using the continuous wave Doppler method, known in the art. In this embodiment, the signal analyzer includes a Doppler signal processor which determines the velocity of blood flow, if any, by measuring a difference in frequency between the reflected signals and the originally transmitted signals.

A binary mode indicator is associated with each transducer. Each indicator, when in an active mode, generates an output reference signal (for example, light or sound), and when in an inactive mode generates no output signal. Each indicator switches onto the active mode only if blood flow is detected from the reflected signals received by its associated transducer, and otherwise remains in the inactive mode. In one embodiment, an activator, such as an ON-OFF switch, may active each indicator if blood flow can be detected from the signals received by the associated transducer.

Instead of a plurality of indicators, the apparatus may include a single signaling device that generates output reference signals to alert the user that the desired bifurcation location has been reached. In this embodiment, the signal analyzer may be configured to vary the characteristics of the output reference signals generated by the signaling device, as a function of the distance between the probe to the bifurcation. For example, the frequency or intensity of light or sound signals, or the tone of a beep, or the color of a light signal, may be varied as a function of the distance between the probe and the bifurcation, thereby allowing the user or technician to easily locate the bifurcation.

In one embodiment, the apparatus may include a dual mode probe, which in a locating mode is operable to determine the location of the bifurcation, and in a measuring mode is operable to measure the thickness of intima media at the bifurcation. In this embodiment, the probe switches into the measuring mode upon determination of the location of the bifurcation, then measures the thickness of intima-media at the bifurcation, using known measurement techniques.

A method of locating a bifurcation of an artery includes moving an array of ultrasound transducers along the artery, while rapidly and sequentially energizing the transducers. Each transducer transmits ultrasound signals onto a portion of the anatomy underneath the transducer, and receives ultrasound signals reflected from the portion of the anatomy. Each transducer has an associated indicator disposed adjacent the transducer.

The method includes analyzing the received ultrasound signals to determine whether blood flow can be detected from the received signals. The method includes causing each indicator to generate an output reference signal only if blood flow is detected from the ultrasound signals received by the associated transducer. The method includes observing the reference signals from the indicators to determine where two reference signals that were initially physically separated from each other merge into a single output signal, or where two physically separated groups of reference signals merge into a single group of output signals, in order to locate the bifurcation.

### BRIEF DESCRIPTION OF THE DRAWINGS

FIG. 1 schematically illustrates an apparatus for locating a bifurcation of an artery, in accordance with one embodiment.

FIGS. 2A, 2B, and 2C illustrate how different indicators are illuminated, as the probe is moved from a location away from the bifurcation, towards and beyond the bifurcation.

FIGS. 3A, 3B, and 3C illustrate an embodiment in which a single signaling device is used, instead of a plurality of indicators. The tone of the acoustic reference signals generated by the signaling device is varied as a function of the distance between the probe and the bifurcation, from a relatively low tone when the probe is located furthest from the bifurcation (FIG. 3A), to a higher tone when the probe is moved closer to the bifurcation (FIG. 3B), and to a highest tone when the probe is moved closest to the bifurcation (FIG. 3C).

FIGS. 4A and 4B schematically illustrate a pulse-echo technique, known in the art, for measuring the thickness of intima media within a vessel.

### DETAILED DESCRIPTION

An apparatus and method for locating a bifurcation of an artery are described. The bifurcation is located by detecting the flow of blood beneath ultrasonic transducers. The apparatus may include an elongated probe, and a signal analyzer. The apparatus is cost efficient, simple to operate, and requires no complex image processing.

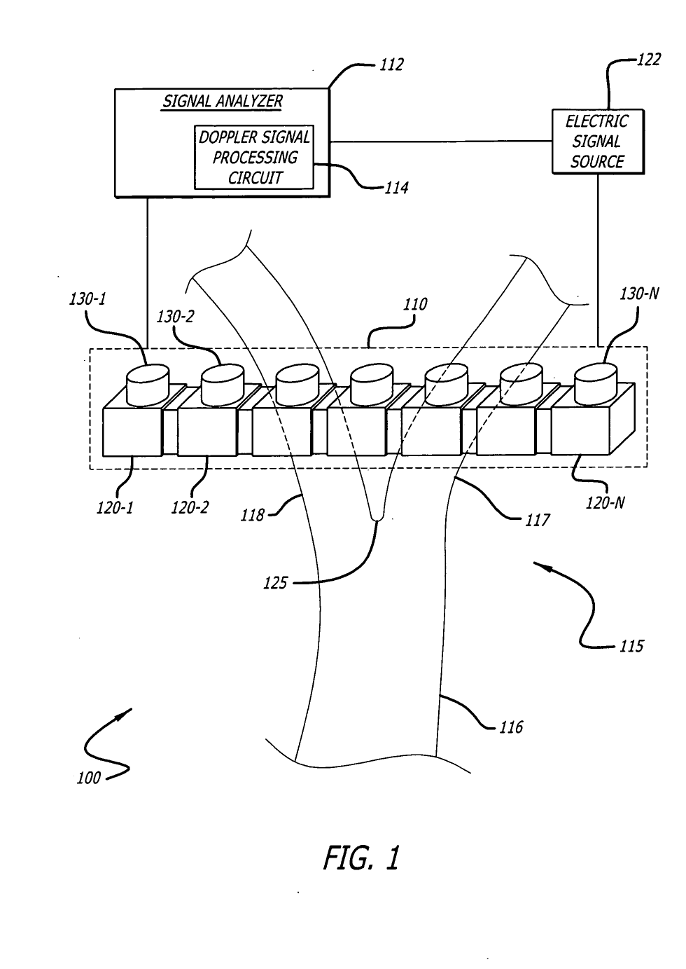

FIG. 1 schematically illustrates an apparatus 100 for locating a bifurcation 125 of an artery 115, in accordance with one embodiment. In the illustrated embodiment, the artery 115, which may be a carotid artery, includes a single branch 116 below the bifurcation 125, and separates into a first branch 117 and a second branch 118, above the bifurcation 125. In overview, the apparatus 100 includes an elongated probe 110, and a signal analyzer 112. The probe 110 has a plurality N of transducers, and an indicator associated with each transducer. In FIG. 1, the plurality N of transducers are shown using reference numerals 120-1, 120-2, . . . , 120-N, and the associated indicators are shown using reference numerals 130-1, 130-2, . . . , 130-N. In the illustrated embodiment, the plurality N of transducers 120-1, . . . , 120-N are disposed and arranged in an array. A substantially linear array is shown in FIG. 1, however other embodiments may include different types of arrangements of the transducers, including but not limited to non-linear arrays of transducers.

In one embodiment, the transducers 120-1, . . . , 120-N are ultrasound crystals that generate ultrasonic frequency vibratory signals, upon receipt of electric energy from an electric signal source 122, and transmit these ultrasonic signals onto a medium, e.g. onto anatomical tissue under examination. In particular, each transducer transmits the ultrasound signals that it generates onto a portion of the anatomy located directly underneath the transducer. In the illustrated embodiment, the transducers are also operable in a receiving mode, in which each transducer receives “echo” signals, i.e. signals that have been reflected from the tissue or other medium.

In the illustrated embodiment, the indicators 130-1, . . . , 130-N are binary mode indicators, having an active mode and an inactive mode. Each indicator is switched into the active mode only if the reflected echo signals show the presence of blood flow within the medium from which they were reflected. In the active mode, the indicator generates one or more output reference signals, alerting the user that blood flow has been detected from the echo signals received by the transducer associated with the indicator. In the inactive mode, no output signal is generated by the indicator.

The echo signals received by the transducers are converted into electric signals by the transducers 120, and then sent to the signal analyzer 112 to be processed and analyzed. In one embodiment, the signal analyzer 112 includes signal processing circuitry which processes the echo signals to determine whether any blood flow can be detected within the tissue from which the echo signals have been reflected. Detecting blood flow within a medium by analyzing ultrasound signals that have been reflected from the medium is a process well known in the art. Any known technique may be used by the signal analyzer 112 to determine whether blood flow is present. These techniques include, but are not limited to, continuous wave Doppler method, or de-correlation analysis between temporal adjacent echoes, which is based on the theory that echoes from blood show little correlation among themselves.

In the embodiment illustrated in FIG. 1, the Doppler method is used, and the signal analyzer 112 includes a Doppler signal processing circuit 114. As well known, the Doppler method for measuring blood flow involves transmitting an ultrasonic signal of a known frequency onto a medium under examination, e.g. anatomical tissue, and analyzing the ultrasonic echo that is reflected from the medium. Since blood flows with a certain velocity, the echo reflected from the particulates of the flowing blood includes a Doppler frequency shift that is related by a well known relationship to the blood flow velocity, the velocity of sound, and the known frequency of the ultrasonic signal that was transmitted onto the medium.

In this embodiment, the Doppler signal processing circuit 114 extracts Doppler frequency shift components, if any, from the electric signals that were sent by the transducers 130-1, . . . , 130-N and that are representative of the reflected echo signals. In particular, the Doppler signal processing circuit 114 measures the frequency of the echo signal, and computes the difference, if any, between the frequency of the echo signal and the known frequency of the original ultrasonic signals transmitted by the transducers onto the medium. If any such Doppler frequency shift is detected, a control signal may be sent by the Doppler signal processing circuit 114 to the indicator associated with the transducer that received the echo signals, so that the indicator is activated, i.e. is switched into the active mode. The Doppler signal processing circuit 114 may also compute the velocity of blood flow detected by the echo signals, using an appropriate arithmetic circuit and/or divider circuit.

In the illustrated embodiment, the transducers 120 are arranged within the probe 110 so as to form an array of transducers. A seven-transducer array is shown for illustrative purposes, i.e. N=7 in the illustrated embodiment; however different embodiments may use different numbers of transducers. Typically, the total number of transducers may vary from about 6 to about 10, although a probe having a number of transducers that is outside a 6 to 10 range is also within the scope of the present invention.

In one embodiment, the output reference signals may be visual signals, for example light signals, and the indicators are indicator lights that become illuminated when switched into the “ON” mode. In this embodiment, each indicator becomes illuminated when the echo signals from its associated transducer, when analyzed, show the presence of blood flow. The indicators may be LCDs, for example, or any other type of light emitters known in the art. Other embodiments may use different types of reference signals that alert the user that blood flow has been detected from the echo signals received by its associated transducer and analyzed by the signal analyzer 112. These different types of reference signals may include, but are not limited to, acoustic signals. Preferably, each indicator is disposed adjacent its associated transducer. Each indicator may be disposed just above its associated transducer, as shown in FIG. 1, or may be disposed just below its associated transducer.

In one embodiment, the apparatus 100 may include an activator (not shown in FIG. 1) that activates each indicator, if and only if the echo signals received by the transducer associated with the indicator show the presence of blood flow within the medium from which the signals were reflected. In one embodiment, the activator may be a simple ON-OFF switch for turning the indicators on or off. In this embodiment, the activator turns on a particular indicator, if the activator receives a control signal from the Doppler signal processing circuit indicating the presence of blood flow within the medium from which the echo signals received by the transducer associated with that indicator were reflected.

The apparatus 100 is not limited, of course, for use only with an artery (illustrated in FIG. 1) that has one branch below the bifurcation and two branches above the bifurcation. Rather, the apparatus 100 can be used to locate the bifurcation of any artery that has a single branch on one side of the bifurcation, and that separates into at least a first branch and a second branch on the other side of the bifurcation.

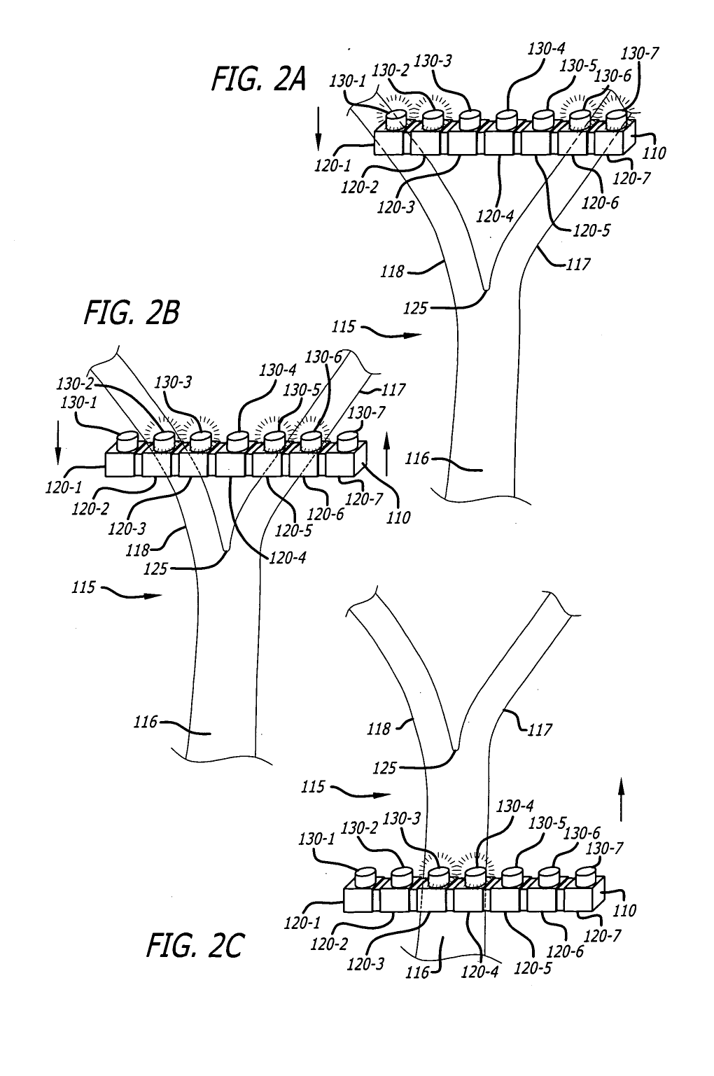

FIGS. 2A, 2B, and 2C illustrate how different indicators within the probe 110 are activated, as the probe 110 is moved from a location away the bifurcation 125, towards the bifurcation, then further beyond the bifurcation. FIGS. 2A, 2B, and 2C graphically show that two outer segments of indicator lights in the array are illuminated, when the probe 110 is above the bifurcation and relatively far from the bifurcation, and that the two illuminated segments of indicator lights move closer together until, eventually, they join into a single illuminated segment.

The probe 110 may be moved either up or down the artery 115, which in the embodiment illustrated in FIGS. 2A-2C includes a single branch below the bifurcation 125, and separates into a first branch 117 and a second branch 118 above the bifurcation 125. The plurality of transducers are positioned and arranged within the probe 110 so that when the probe 110 is positioned above the bifurcation 125, the plurality of transducers span both the first branch 117 and the second branch 118, and when the probe 110 is positioned below the bifurcation 125, the plurality of transducers span only the single branch 116. In this way, blood flow can be detected by the Doppler signal processing circuit 114 from both branches 117 and 118 when the probe 115 is positioned above the bifurcation 125, and blood flow can be detected from only the single branch 116 when the probe 115 is positioned below the bifurcation.

The transducers are positioned and arranged so that when the probe 110 is positioned above the bifurcation 125 (as illustrated in FIGS. 2A and 2B), two physically separated output signals, or two physically separated groups of output signals are generated by the indicators. Two physically separated output signals are generated by the indicators, when only one transducer receives echo signals reflected from the branch 117 and only one transducer receives echo signals reflected from the branch 118. Two physically separated groups of output signals are generated, if more than one transducer receives echo signals reflected from the branch 117, and more than one transducer receives echo signals reflected from the branch 118.

The transducers are positioned and arranged so that when the probe 110 is positioned below the bifurcation 125, only a single output signal, or a single group of output signals are generated. A single output signal is generated, when only one transducer receives echo signals reflected from the single branch 116 below the bifurcation. A single group of output signals is generated, when more than one transducer receives echo signals reflected from the branch 116, as illustrated in FIG. 2C.

As illustrated in FIG. 2A, in the beginning the probe is located away from the bifurcation 125. In one embodiment, the transducers may be rapidly and sequentially energized, i.e. the transducers may work alternatively to determine if there is blood flow within a portion of the anatomy that is located directly beneath each transducer. As explained earlier, the indicator associated with each transducer is lighted if and only if blood flow information is detected from the echo signals received by its associated transducer.

When the probe is initially disposed at a location above the bifurcation 125 and is relatively far from the bifurcation, as illustrated in FIG. 2A, the lighted indicators 130-1, 130-2, 130-5, and 130-6 show a two-group pattern, because no blood flow is detected by the transducers 120-3, 120-4, and 120-5 that are located at or near the center of the array of transducers. As seen from FIG. 2A, the lighted indicators are associated with transducers located at or near the ends of the probe 110

As the probe 110 is moved downward toward the bifurcation and approaches the bifurcation 125, however, the two-group pattern gradually becomes narrower, as illustrated in FIG. 2B. In FIG. 2B, the illuminated indicators are 130-2, 130-3, 130-5, and 130-6, i.e. the illuminated segments of light have moved closer together.

Eventually, the two-group pattern merges into a one-group pattern, as shown in FIG. 2C. In FIG. 2c, only the indicators located at the center of the array, namely indicators 130-3 and 130-4, are illuminated. The merging of the indicator signals, from a two-group pattern to a one-group pattern, notifies the user that the probe 110 is located exactly at the bifurcation 125.

By analogy, when the probe starts at a location below the bifurcation 125, and is moved upward, the lighted indicators show a one-group pattern since there is blood flow under the transducers located at or near the center of the array. As the probe approaches the bifurcation 125, the one-group pattern gradually becomes wider, and eventually divides into a two-group pattern that shows that the probe is located above the bifurcation. The direction of movement can then is reversed, so as to position the probe at the point where single flow pattern first appeared.

The probe 110 therefore permits an operator or technician to easily locate the bifurcation of an artery (such as the carotid artery), simply by moving the probe 110 up or down the carotid. As the probe 110 is moved, the indicator attached to each transducer will become illuminated, when blood flow is detected beneath the transducer associated with the illuminated indicator. Above the bifurcation, the lighted indicators show a two-group pattern. Below the bifurcation, the lighted indicators show a one-group pattern. The probe 110 costs a lot less than probes that use ultrasound imaging, because no complex image processing circuitry is required. Also, the probe 110 is simple and easy to use, requiring no skilled or trained technicians for its use.

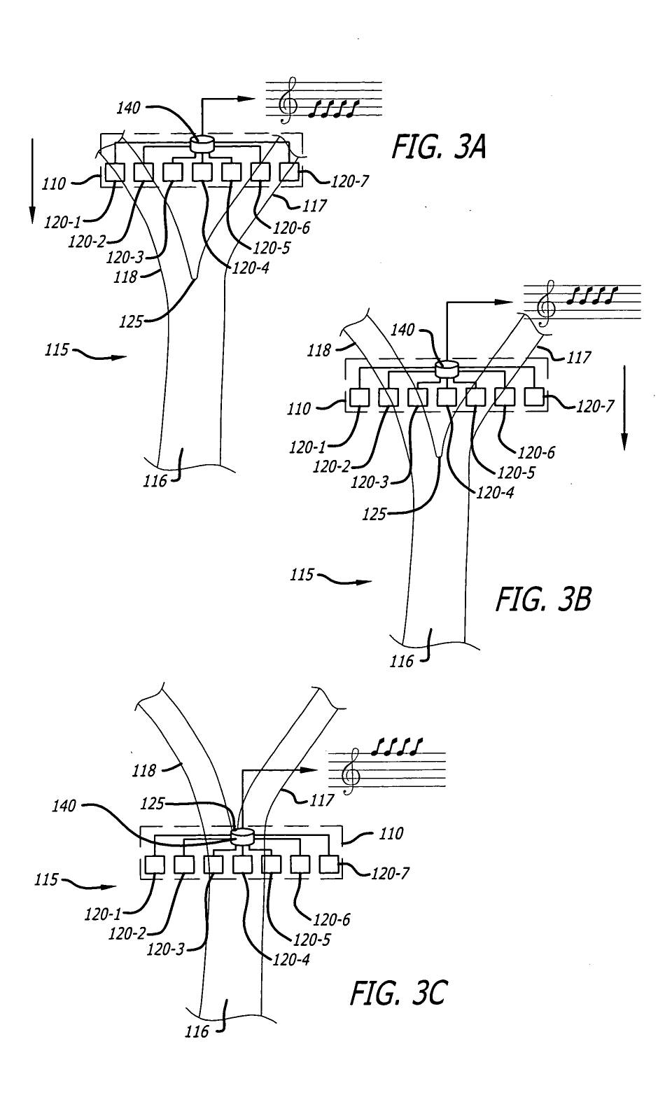

FIGS. 3A, 3B, and 3C illustrate an embodiment in which the probe 110 includes a single signaling device 140 for alerting the user when the probe is has located the bifurcation of the artery, instead of a plurality of indicators attached to associated transducers. The signaling device 140 emits reference output signals, including but not limited to light signals or sound signals, which notify the user that the correct bifurcation location has been reached. In the embodiments illustrated in FIGS. 3A, 3B, and 3C, the signals from each transducer are processed by the signal analyzer 112, to cause the signaling device 140 to alert the operator when the probe is positioned at the desired bifurcation 125 or junction of the “Y.”

This alerting is accomplished by causing a change in the characteristics or type of output signals that are emitted by the signaling device, as the probe approaches the desired location. In this embodiment, the signal analyzer 112 causes the signaling device 140 to vary one or more characteristics of the output reference signals as a function of the proximity of the probe to the bifurcation, i.e. as a function of the distance between the probe and the bifurcation. For example, the frequency of flashes or beeps, the intensity of light or sound, the tone of a beep, or the color of light may be varied as a function of the proximity of the probe to the “Y” junction, thus allowing the technician to quickly zero in on the correct location.

In the embodiment illustrated in FIGS. 3A, 3B, and 3C, the output reference signals from the signaling device 140 are acoustic signals, and the tone of the acoustic signals is progressively varied, from a low note when the probe 110 is located relatively far from the bifurcation 125 (as shown in FIG. 3A), to a somewhat higher note when the probe 110 is moved closer to the bifurcation 125 (as shown in FIG. 3B), and to a highest note when the probe 110 is moved closest to the bifurcation 125 (as shown in FIG. 3C).

In one embodiment, the probe may be a dual mode probe. In this embodiment, the probe can determine the location of the bifurcation when operated in a locating mode, and can measure the thickness of intima media at the bifurcation, when operated in a measuring mode. In this embodiment, once the desired location is reached, the mode of the probe may be switched to the measuring mode, in which the probe measures the thickness of the intima-media, in accordance with existing prior art techniques.

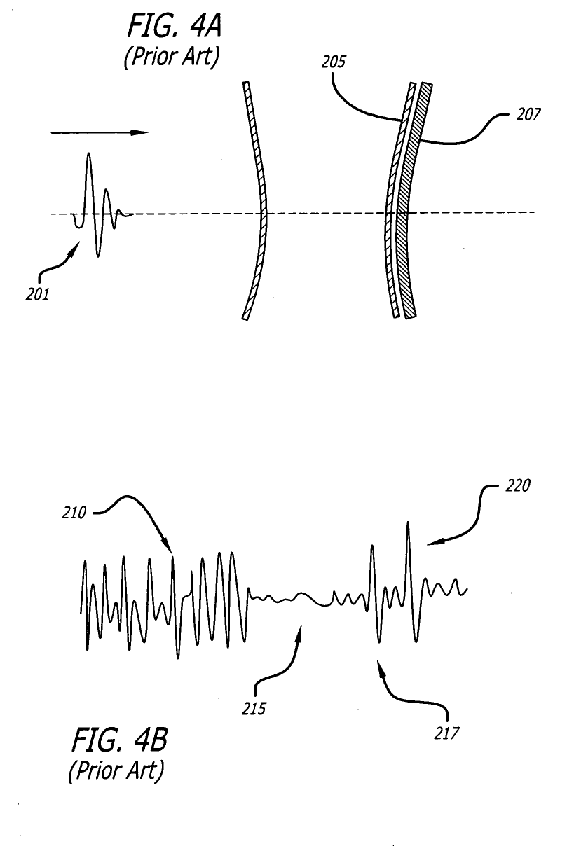

FIGS. 4A and 4B schematically illustrates a pulse-echo technique, known in the art, for measuring the thickness of intima media within a vessel. Once the probe is at the bifurcation, and switched into the measuring mode, one or more of the plurality of transducers, for example a transducer located at or near the center of the linear array, may be used to measure the thickness of the intima media 205. In the embodiment illustrated in FIG. 4A, the transducer works at a pulse-echo mode, i.e. the transducer sends an ultrasound pulse 201, then receives an echo. Along the path of the propagation of the ultrasound pulse 201, the reflected echo is determined by the acoustic mismatch at each interface. The distance D from the interface to the transducer surface can be calculated by measuring the time T at which the corresponding echo was received by the transducer, as measured from the time the pulse 201 was sent by the transducer, using the following equation:
D=T·c/2, (1)
where c denotes the speed of sound in the tissue or other medium from which the echo was reflected.

As shown in FIG. 4B, when the ultrasound pulse 201 is transmitted toward a portion of the carotid artery, the received echo line can be characterized by four parts: echo 210 from the tissue, echo 215 from the blood, echo 217 from the intima, and echo 220 from the rear wall of the vessel. The thickness of the intima media 207 can be calculated from equation (1), by measuring the temporal interval between the echo peak of intima and the rear wall, with a minor correction. As seen from FIG. 4B, the first peak is from the intima media, and the second peak is from the rear wall. The thickness of the intima media 207 is related to the distance between the two peaks.

To locate the echo in the echo line, it can be expedient to first locate the echo from blood. This can be achieved by using the pulsed Doppler mode. By changing the depth of the sampling volume and searching the peak flow, the center of the artery can be determined, as known in the art. Alternatively, the transducer may work at an M-mode, transmitting the ultrasound pulse repeatedly, and determining the center of the blood vessel by de-correlation analysis of the adjacent echoes. Once the vessel center is found, the element can then be switched to pulse-echo mode, and the rear wall of the carotid artery can be located by performing a search from the center of the artery. Previous studies have shown that this approach is workable, since the echo from the intima is quite different from that of the blood.

While the ultrasound probe has been described and shown with reference to specific embodiments, it should be understood by those skilled in the art that various changes in form and detail may be made therein. Many other embodiments are possible.

Other embodiments are within the following claims.

1. An apparatus for locating a bifurcation of an artery, the artery having a single branch below the bifurcation and separating into at least a first branch and a second branch above the bifurcation, the apparatus comprising:
an elongated probe including a plurality of ultrasound transducers configured to generate ultrasound signals when provided with electric energy, the transducers being further configured to transmit the ultrasound signals onto a medium, and to receive reflected signals that have been reflected from the medium;
a signal analyzer configured to analyze the reflected signals received by each transducer so as to determine whether blood flow within the medium can be detected from the received signals; and
a binary mode indicator associated with each transducer, each indicator when in an active mode generating an output reference signal, and when in an inactive mode generating no output signal;
wherein each indicator is configured to operate in the active mode only if blood flow is detected from the reflected signals received by its associated transducer, and to otherwise remain in the inactive mode.

2. An apparatus in accordance with claim 1, wherein the plurality of ultrasound transducers are positioned and arranged within the probe so that when the probe is positioned above the bifurcation, blood flow is detected from both the first and second branches of the artery upon transmission and reception of ultrasonic signals by the transducers, and when the probe is positioned below the bifurcation, blood flow is detected from the single branch below the bifurcation.

3. An apparatus in accordance with claim 1, wherein the plurality of ultrasound transducers are positioned within the probe so as to cause at least two physically separated output reference signals to be generated by the indicators when the probe is positioned above the bifurcation, and only a single output reference signal to be generated when the probe is positioned below the bifurcation.

4. An apparatus in accordance with claim 1, wherein the plurality of ultrasound transducers are positioned within the probe so as to cause at least two physically separated groups of output reference signals to be generated by the indicators when the probe is positioned above the bifurcation, and only a single group of output reference signals to be generated when the probe is positioned below the bifurcation.

5. An apparatus in accordance with claim 1, wherein the plurality of transducers comprise a substantially linear array of transducers, and wherein each indicator is disposed adjacent its associated transducer.

6. An apparatus in accordance with claim 1, wherein the signal analyzer comprises a Doppler signal processor configured to extract from the reflected signals any Doppler frequency shift components within the reflected signals, by computing a difference between the frequency of the received reflected signal and the frequency of the transmitted signal.

7. An apparatus in accordance with claim 1, wherein the indicators comprise light indicators, and wherein the output reference signals generated by each indicator comprise light signals.

8. An apparatus in accordance with claim 7, wherein the indicators comprise LCDs.

9. An apparatus in accordance with claim 2, wherein the transducers are arranged so that the indicators associated with transducers located at or near the ends of the probe become activated when the probe is above the bifurcation and relatively far from the bifurcation, and so that the indicators associated with transducers disposed at or near the center of the probe become activated when the probe approaches the bifurcation.

10. An apparatus in accordance with claim 9, wherein the transducers are positioned and arranged within the probe so that when the probe is moved from a location away from the bifurcation in a direction toward the bifurcation, the distance between two physically separated reference signals or two physically separated groups of reference signals progressively decreases, until the two reference signals or the two groups of reference signals merge at the bifurcation into a single reference signal or a single group of reference signals, respectively.

11. An apparatus in accordance with claim 5, wherein each indicator is disposed directly above its associated transducer.

12. An apparatus in accordance with claim 5, wherein each indicator is disposed directly below its associated transducer.

13. An apparatus in accordance with claim 1, further comprising an electric signal source configured to supply electric energy to the transducers.

14. An apparatus for locating a bifurcation of an artery, the artery having a single branch below the bifurcation and separating into at least a first branch and a second branch above the bifurcation, the probe comprising:
a probe including a plurality of ultrasound transducers configured to transmit ultrasonic signals onto a medium, and to receive ultrasonic signals reflected from the medium;
a signal analyzer configured to analyze the ultrasonic signals received by each transducer so as to determine whether blood flow within the medium can be detected from the received signals; and
a signaling device configured to generate one or more output reference signals upon detection of blood flow by the signal analyzer;
wherein the plurality of ultrasound transducers are positioned and arranged so that when the probe is above the bifurcation, the transmission and reception of ultrasonic signals by the transducers cause blood flow detection from both the first and second branches, and when the probe is below the bifurcation, the transmission and reception of ultrasonic signals by the transducers cause blood flow detection from only the single branch below the bifurcation; and
wherein the signaling device is configured to vary a characteristics of the output reference signals as a function of the distance between the probe and the bifurcation.

15. An apparatus in accordance with claim 14, wherein the output reference signals comprise acoustic signals, and the characteristics of the output signals that is varied comprises one of a frequency and an intensity of the acoustic signals.

16. An apparatus in accordance with claim 14, wherein the output reference signals comprise visual signals, and the characteristics of the output signals that is varied comprises one of a) a frequency of the visual signals; b) an intensity of the visual signals; and c) a color of the visual signals.

17. A dual mode probe for measuring intima media thickness at a bifurcation of an artery, the artery having a single branch below the bifurcation and separating into at least a first branch and a second branch above the bifurcation, the probe being operable in a locating mode to determine the location of the bifurcation, and in a measuring mode to measure the thickness of intima media at the bifurcation, the probe comprising:
an array of ultrasound transducers, each transducer configured to generate ultrasonic signals when provided with electric energy, each transducer being further configured to transmit the ultrasonic signals onto a medium, and to receive ultrasonic signals reflected from the medium;
a signal analyzer configured to analyze the signals received by each transducer, the signal analyzer when in the locating mode being configured to determine whether blood flow can be detected within the medium from which the received signals have reflected, the signal analyzer when in the measuring mode being configured to measure the thickness of intima media at the bifurcation by detecting from the received signals a first peak corresponding to a reflection from a portion of the intima media, and a second peak corresponding to a reflection from a portion of the wall of the artery behind the intima media, and measuring the distance between the first peak and the second peak; and
a binary mode indicator associated with each transducer, each indicator when in an active mode generating a visible output signal, and when in an inactive mode generating no output signal, each indicator operating in the active mode only if blood flow is detected from the signals received by its associated transducer, and otherwise remaining in the inactive mode;
wherein when the probe in the locating mode is positioned above the bifurcation, two physically separated signals or two physically separated groups of signals are generated from the indicators because of blood flow detection from both the first and second branches, and when the probe in the locating mode is positioned below the bifurcation, a single signal or a single group of signal is generated from the indicators because blood flow is detected only from the single branch below the bifurcation; and
wherein upon detection of the bifurcation, the probe switches into the measuring mode, so that the signal analyzer can determine the thickness of intima-media.

18. A method of locating a bifurcation of an artery, the artery having a single branch below the bifurcation and separating into at least a first branch and a second branch above the bifurcation, the method comprising:
moving a substantially linear array of ultrasound transducers down the artery while sequentially energizing the transducers so that the transducers transmit ultrasound signals onto one or more portions of the anatomy at or near the artery, and receive ultrasound signals reflected from the one or more portions of the anatomy, each transducer having an associated indicator disposed adjacent the transducer;
analyzing the received reflected signals to determine whether blood flow can be detected from the received signals;
causing each indicator to generate an output reference signal only if blood flow is detected from the ultrasound signals received by the transducer associated with the indicator; and
observing the reference signals from the indicators to determine where two physically separated reference signals merge into a single reference signal, or where two physically separated groups of reference signals merge into a single group of output signals, to locate the bifurcation.
